(19) United States
(12) Patent Application Publication
(10) Pub. No.: US 2003/0195431 A1
(43) Pub. Date: Oct. 16, 2003

(54) Observation of effects of body fluids on indicator cells for disease diagnosis

(76) Inventor: Vikas P. Sukhatme, Newton, MA (US)

Correspondence Address: CLARK & ELBING LLP, 101 FEDERAL STREET, BOSTON, MA, 02110, US

(21) Appl. No.: 10/120,022

(22) Filed: Apr. 10, 2002

(51) Int. Cl. A61B 10/00

(52) U.S. Cl. 600/562

(57) ABSTRACT

The invention features methods of obtaining information regarding a health parameter of a patient, in which a cell-free body fluid is incubated with an indicator cell and changes in the indicator cell, such as an increase or decrease in gene expression are observed.

### BACKGROUND OF THE INVENTION

Many diagnostic methods are currently used to diagnose disease and monitor the efficacy of the treatment of disease. A number of these methods examine the change in gene expression level in certain cells brought about by the disease. This is most commonly carried out by extracting the RNA from a diseased cell and applying it to a gene chip, e.g., those sold by Affymetrix and described in the Affymetrix patents e.g., U.S. Pat. No. 6,344,316, and observing an altered pattern of gene expression.

### SUMMARY OF THE INVENTION

My invention is premised on the assumption that a disease, either directly or via the response of the patient to the disease, will cause a change in the composition of a body fluid, e.g., urine, blood, or lymph fluid. When the fluid is contacted with a living cell, the altered composition of the fluid can bring about a change in gene expression of the cell, or other cellular changes, such as the production of molecules (e.g., lipids), or changes in post-translational modifications of proteins (e.g., alterations in glycosylation patterns). Thus, in one aspect, the invention examines, for diagnostic and other purposes, not the gene expression pattern of the cells of a patient, but rather the gene expression pattern or other changes in non-patient cells that have been contacted with a substantially acellular body fluid of the patient, which is presumed to contain molecules that affect gene expression in the contacted cell.

Accordingly, the invention features, in one aspect, a method for obtaining information regarding a health parameter of a patient, by the steps of: a) obtaining a sample of a substantially cell-free body fluid from the patient, b) incubating the sample with a cell, and c) determining the level of expression of one or more genes in the cell following incubation. When the method is used to monitor the progress of a disease, steps a)-c) are repeated at a later time point, and the results of the two time points are compared to determine whether the gene expression pattern has altered over time. This embodiment of the method is particularly important where, between the two time points, the patient has been treated, and the efficacy of the treatment is to be evaluated; an alteration in gene expression can be used as an indicator of treatment efficacy, or treatment toxicity.

Multiple cell types can be used, in the form of a panel, for testing against any given sample of body fluid. The effect of the fluid on the expression of a single gene can be determined, or the pattern of expression of multiple genes can be observed.

In another aspect of the invention, also taking advantage of the central premise described above, the sample is incubated with genetically engineered cells containing a molecule that is responsive to a substance whose presence or absence in a body fluid is associated with a particular disease state. The response of that molecule in the engineered cell to the body fluid provides information with respect to the disease state of the patient from whom the body fluid sample was obtained. A variety of responsive molecules can be employed in such engineered cells, including receptors and transcription factors; preferably, these are associated with a reporter molecule that generates a signal when a substance in the body fluid interacts with the responsive molecule in the engineered cell.

In another aspect, the invention features a method of making a disease-specific array (DSA) for use in the diagnosis or monitoring of a predetermined disease, by the steps of: a) obtaining a sample of a substantially cell-free body fluid from a patient suffering from the disease, b) incubating the sample with a cell, c) determining the level of expression of multiple genes in the cell following incubation, d) comparing the information contained in step c) with the expression level of multiple genes observed when the same cell type is incubated, under the same conditions, with a sample of the same body fluid obtained from a person suffering from that disease, to identify one or more genes that are expressed at different levels in the two samples, and e) incorporating the genes identified in step d), or RNA molecules corresponding to the genes, or fragments of the genes, into or onto a carrier to form the DSA.

In a further aspect, the invention features a method of obtaining a gene expression signature specific for a predetermined disease by the steps of: a) obtaining a sample of a substantially cell-free body fluid from a patient suffering from the disease, b) incubating the sample with the cell, and c) determining the level of expression of multiple genes following incubation, wherein that determination constitutes the disease-specific signature.

As will be made clear from the discussion that follows, the invention can employ a wide variety of indicator cells (ICs). It is not essential that these ICs mimic in every detail all of the various cell types of the body, nor is it necessary that they contact the acellular body fluid under conditions that are representative of conditions that exist in the body. It is also not necessary that ideal conditions be used for the culturing of the ICs, pre- and post-contact with a body fluid. More important is standardization of conditions under which the ICs are cultured and tested against body fluids, so that any changes in gene expression that are observed have a high probability of being a function of the interaction between the IC and one or more substances in the body fluid, rather than a variation in the culture or the contacting conditions.

As is mentioned above, in some instances a body fluid will be contacted with more than one different IC to obtain more complete information on the changes in the composition of the body fluid caused by a particular disease. It is envisioned that a physician or medical care provider will have available a panel of multiple ICs, and will use a subset of that panel in a given situation, e.g., a medical practitioner may choose a specific subset of ICs when monitoring or If diagnosing bladder cancer using urine.

The methods of the invention offer a number of important advantages. First, because it uses cells of the IC panel rather than cells of the patient whose gene expression is observed, the number of different types of cells that can be used is greatly increased. Furthermore, the methods offer the prospect of much more informative and subtle readouts than traditional gene profiling. To illustrate this principle, assume that there exists a single nucleotide polymorphism (SNP) in a non-coding regulatory region of a gene, and that the SNP results in a decreased transcription rate of the gene, resulting in decreased expression of the encoded protein, resulting in turn in lower blood concentrations of the protein. This lower protein concentration within the body fluid, i.e., blood, can be detected according to the invention if it changes the gene expression pattern in an IC. Similarly, if a SNP occurred in a coding region, and thus affected protein structure/function, this is also detectable according to the invention, as the IC will respond to the altered protein by exhibiting an altered gene expression pattern. The methods of the invention can also detect post-translational alterations in proteins, as these also can result in altered protein function, and thus cause altered gene expression in ICs. Further, the approach of the invention is sensitive not only to proteins in the body fluid being tested, but to any substance present in the fluid, including lipids, small molecules, and carbohydrates, all of which can cause changes in gene expression in ICs.

Another important advantage of the methods of the invention is that they measure an integrated, or net, response to factors that, together, influence gene expression in ICs, and there is thus no need to define the contribution to that net effect by each individual factor in the body fluid. This “integrative” readout is more informative and more consistent with the complexity of biological systems than assays that measure the effect of only one factor. For example, it is known that VEGF levels in the blood correlate roughly with prognosis in a number of types of cancers that are characterized by neovascularization. However, there is no question but that tumor cells produce additional pro-angiogenic agents of which we are unaware, and which cannot be taken into account by simply measuring VEGF levels. According to the methods of the invention, all factors that effect angiogenesis, both angiogenic factors and anti-angiogenic factors, are necessarily taken into account by determining the net effect of all of the factors, known and unknown, on a responsive IC, e.g., an endothelial cell. Thus, the methods of the invention provide a much more informative and true picture of the net effect of all of the factors on the gene expression pattern of an IC, something which is not possible with current single-molecule assay systems.

In carrying out the methods of the invention, it is important to employ suitable controls, so that potential “biological background noise” caused by such issues as diurnal variation, variation with levels of activity and excitement, and wake or sleeping state, do not confound the results. Thus, for example, if urine samples are taken from a patient before, during, and following treatment, the samples should be obtained at the same time of day, and under other similar circumstances.

In addition to providing sensitive methods for monitoring the response of a patient to a treatment being administered for a particular disease, as well as providing a means for early detection of disease, the methods of the invention offer additional advantages, including the opportunity to gain knowledge about the mechanistic process of a particular disease. For example, the IC gene expression data generated over time from the monitoring of multiple patients with a particular disease will facilitate the identification of previously unknown factors in body fluids that influence changes in gene expression patterns in ICs characteristic of the disease. This, in turn, can lead to the identification of new targets for drug discovery, and the testing of drugs against these targets to determine if the proposed drugs alter gene expression in ICs in a beneficial way.

### DETAILED DESCRIPTION

As is discussed above, the determination of the expression level of multiple genes of an indicator cell (IC) following incubation with a cell-free body fluid has a number of applications, which will be discussed in greater detail, following a discussion of the components of the systems of the invention.

Indicator Cells

For some of the diagnostic and treatment-monitoring applications of the invention, there will be provided a panel of multiple different IC types, each of which has a phenotype that will provide a gene expression pattern readout that is appropriate for one or more acellular body fluids to be tested and/or one or more disease states to be diagnosed or monitored. In any given assay, a body fluid can be incubated with several, e.g., 3 to 7, different ICs of the panel; the use of the multiple ICs provides more gene expression information than can be obtained from a single IC.

1. Endothelial Cells

These cells are suitable ICs where the body fluid potentially contains substances that influence angiogenesis. Disease states that potentially contribute to the appearance of such substances in body fluids include vascularized neoplasms, diabetes, and certain ophthalmic disorders, e.g., macular degeneration, that are characterized by neovascularization. The body fluid to be tested against such cells can be urine (e.g., if bladder cancer is suspected or being monitored), blood, tumor-related ascites fluid, lymph fluid, or aqueous humor. The endothelial cells can be immortalized as well as primary cells, and can be derived from various beds, e.g., microvascular, arterial, and venous. Microvascular cells can be of a variety of origins, e.g., lung, dermal, etc.

2. Epithelial Cells

These cells are suitable ICs where the body fluid potentially contains substances that influence ion transport across cell membranes; these ICs are used, e.g., to detect altered gene expression patterns caused by substances present in body fluids obtained from patients with heart and kidney disease, and by substances that are present in carcinomas (cancers of epithelial cell origin), e.g., growth factors such as insulin-like growth factor, differentiation factors such as transforming growth factor-beta, and hormones such as luteinizing hormone. The body fluid can be urine (e.g., if bladder cancer is being monitored), blood, ascites fluid, and/or lymph fluid.

3. Lymphocytes

These cells are suitable ICs where the body fluid potentially contains substances that influence the immune system and/or are indicators of immune system dysfunction, e.g., cytokines and antibodies against healthy tissue. The body fluid contacted with lymphocytes can be blood or lymph fluid, and the disease state detected or monitored can be any immune system disorder, such as an autoimmune disease, e.g., rheumatoid arthritis and type-1 diabetes; graft- vs. host disease; allograft rejection; leukemia; or lymphoma.

4. Excitable Cells

There are a number of excitable cells, including neurons and contractile muscle cells, that can serve as ICs to monitor the “excitable environment” (i.e., compounds that interact with excitable cells to change their gene expression patterns) in body fluids such as blood. Changes in gene expression patterns in such cells following contact with a blood sample from a patient can be indicative of neurologic disorders such a Parkinson's Disease and muscular and neuromuscular disorders such as Multiple Sclerosis and Amyotrophic Lateral Sclerosis.

5. Embryonal Carcinoma Cells

These cells are suitable ICs for testing virtually any body fluid, and the gene expression patterns exhibited by these cells can be indicative of virtually any disease (including carinomas), as these cells are known to respond to many diverse compounds, including proteins and polysaccharides.

6. Embryonic Stem Cells

These cells, like embryonal carcinoma cells, can be used as ICs to test any body fluid and/or to detect or monitor a wide variety of molecules.

7. Stem Cells from Non-Embryonic Sources

Stem cells obtained from adult humans (e.g., from blood or bone marrow) and stem cells obtained from umbilical cord blood can be used in the same circumstances as described above with respect to embryonic stem cells, as these cells also respond to a wide variety of stimuli.

8. Endothelial Precursor Cells for Angiogenesis

These cells, which are less differentiated than mature endothelial cells, but more differentiated than stem cells, can serve as ICs in a number of circumstances, using the body fluids discussed above. The gene expression patterns induced in these cells upon incubation with a particular body fluid may differ from the patterns observed in stem cells and highly differentiated cells.

9. Highly Specialized Cells

In some instances, e.g., where the fluid, such as urine or blood, of a patient, is likely to contain endocrine factors, it will be useful to employ, as an IC, a specialized cell that is likely to respond, in its pattern of gene expression, to such factors. For example, Islet cells can serve as ICs for the testing of blood for early indications of Type I diabetes. Another example is the use as ICs of cells of the human cerebral cortex, which can be tested against cerebrospinal fluid for early detection of Alzheimer's Disease.

10. Non-Human Cells

In some cases it may be possible to use non-human vertebrate cells, e.g., cells from other mammals such as chimpanzees or swine, particularly in cases where specialized cell lines of human origin are unavailable. Cells from other vertebrates such as zebrafish or Xenopus can also be used in some situations, and collections of cells from such animals, e.g., early-stage embryos, can also be used. In some instances, other eukaryotic cells can be used, even if they are not derived from vertebrates; an example is yeast, which share a number of conserved genes with humans and other mammals. All of these cell types can be used in their native state, or they can be genetically engineered as described below.

11. Engineered Cells

As is mentioned above, in some cases it will not be necessary to determine the level of expression of multiple genes in an IC following incubation of the IC with a body fluid. An alternative method that can be used in some instances is to examine the effect of the body fluid on one gene or other responsive molecule in a genetically engineered cell. For example, if the body fluid is obtained from a patient being monitored, diagnosed, or treated for a particular hormone-dependent cancer, e.g., a cancer of the female reproductive system that results in the over-production of a peptide fertility hormone, such as follicle stimulating hormone (FSH), which would be present in the urine of the patient in increased concentrations if the patient were suffering from one of these cancers, the IC could be a human cell, of any type, e.g., a fibroblast, engineered to bear on its surface the receptor for FSH. Preferably, the gene encoding the responsive molecule is part of a reporter construct, including a reporter gene such as the lacZ gene encoding the reporter protein beta-galactosidase.

One common method of generating cells that contain the gene reporter constructs mentioned above, or that express the required cell-surface receptor, is by making use of viral vectors, for example, retroviral vectors (see Miller et al., 1993, Meth. Enzymol. 217:581-599; Boesen et al., Biotherapy 6:291-302, 1994; Clowes et al., J. Clin. Invest. 93:644-651, 1994; Kiem et al., Blood 83:1467-1473, 1994; Salmons and Gunzberg, Human Gene Therapy 4:129-141, 1993; and Grossman and Wilson, Curr. Opin. in Genetics and Devel. 3:110-114, 1993), adenovirus vectors (Kozarsky and Wilson, Current Opinion in Genetics and Development 3:499-503, 1993; Rosenfeld et al., Science 252:431-434, 1991; Rosenfeld et al., Cell 68:143-155, 1992; and Mastrangeli et al., J. Clin. Invest. 91:225-234, 1993), adenovirus-associated vectors (AAV; see, for example, Walsh et al., Proc. Soc. Exp. Biol. Med. 204:289-300, 1993), herpes virus vectors, or pox virus vectors. Stable incorporation of the genetic element will provide for an engineered cell that will stably contain or express the genetic information necessary for that cell to act as an IC of the invention. One can also use non-viral methods to introduce genetic information into a preferred cell type. These methods include transformation using naked DNA (typically in plasmid form) delivered via liposomes, receptor-mediated delivery, calcium phosphate transfection, lipofection, electroporation, particle bombardment (gene gun), microinjection, cell fusion, chromosome-mediated gene transfer, microcell-mediated gene transfer, spheroplast fusion, or pressure-mediated gene delivery. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, in a manner such that the necessary developmental and physiological functions of the recipient cells are not disrupted. Usually, the method of transfer includes the transfer of a selectable marker to the cells. The cells are then placed under selection to isolate those cells that have taken up and are expressing the transferred gene. The technique is carried out by known methods that ensure that there is stable transfer of the genetic information to the cell, so that the gene is expressible by the cell and preferably heritable and expressible by its cell progeny.

Engineered cells useful in the methods of the invention can be made based on genetic information obtained from multiple instances of incubating body fluids with ICs. In an illustrative hypothetical example, genes X, Y and Z have been determined to be upregulated when endothelial cells are incubated with fluid from patients with a certain kind of cancer, e.g., ovarian cancer, based on multiple instances of incubating the endothelial cell IC with the cancer fluid and analyzing gene expression changes. To rapidly screen the transcriptional activation of these genes, the promoter enhancer fragments of these genes that respond to the fluid are used to construct three different reporter constructs, one for gene X, in which the gene promoter turns on a first reporter, e.g., alkaline phosphatase; a second construct in which the promoter from gene Y directs the expression of human growth hormone; and a third construct in which the gene promoter directs the expression of the luciferase reporter. These three constructs are transfected individually and sequentially into a fibroblast IC, and the fibroblast IC is then incubated with the ovarian cancer fluid. The reporters provide a readout indicating whether any of the promoters were upregulated upon such incubation.

If the IC being employed, e.g., a fibroblast, does not contain the necessary machinery to receive the incoming signals, for example, a receptor for VEGF, which potentially is the factor in ovarian cancer fluid that activates these genes in endothelial cells by binding to receptors for VEGF, one can transfect into the fibroblast a fourth construct, an expression vector for the VEGF receptor, such that this engineered fibroblast bears on its surface the receptor for VEGF, as well as the three reporter genes known to be upregulated in endothelial cells when ovarian cancer fluid is incubated with them. These engineered cells can serve as a simple read-out for HUVEC gene expression patterns because it is routine to assess the expression of these reporter constructs (i.e., human growth hormone, luciferase, and alkaline phosphate), which can serve as proxies for the more expensive methodology of extracting the RNA from the cell and then hybridizing the RNA to a chip.

Body Fluids

As is discussed above, the fundamental assumption underlying the invention is that the body fluid being tested will contain one or more substances that are elaborated by diseased cells in the body, or by cells in the body in response to the disease. These substances will alter the gene expression responses in the ICs. In instances in which the disease at issue is a systemic one, e.g., one of the leukemias, the blood that is tested against one or more ICs can be obtained from any body location. In other instances, a disease might affect only a few, localized cells, and thus the systemic effect may, at least at an early stage, be too small to permit the meaningful testing of body fluids remote from the disease. Examples are early ovarian cancer, bladder cancer, and diseases of the pleura. In such cases, it is useful to obtain fluids that are in close proximity to the site of the disease. For example, in the case of a patient with ovarian cancer, ascites in the region of the tumor is the preferred fluid. Similarly, urine is the preferred fluid to test for bladder cancer, and pleural effusion is tested where the disease affects the pleura.

As is discussed in detail above in connection with the ICs that can be used in the methods of the invention, the body fluid that is tested against one or more ICs in connection with the diagnosis or monitoring of a particular disease is chosen with two factors in mind: 1) the body fluid should be one which likely contains substances that are elaborated by diseased cells, or by cells in the body that respond to the disease, that will in turn alter gene expression patterns in ICs; and 2) the fluid should be obtained from a locus sufficiently proximate to the diseased tissue, if the disease is local and/or early stage. In addition to the body fluids discussed above, the invention can employ many others, including, without limitation, synovial fluid, peritoneal fluid, semen, breast milk, saliva, and liquified extracts from tissues at particular sites, including samples from tumors and other diseased tissues.

Readouts other than Gene Expression

As is mentioned above, rather than examine changes in gene expression patterns caused by the presence of substances in body fluid samples, for diagnostic and other purposes, one can exam other effects on test cells (ICs) brought about by incubation of the cells with the body fluid of interest.

One important category of changes that can be examined are post-translational changes in proteins expressed by the ICs. Such changes include, for example, changes in phosphorylation (e.g., the degree of phosphorylation, or an increase or decrease in phosphorylation), glycosylation (e.g., an increase or decrease in glycosylation, or a change in glycosylation pattern), lipid attachment, sulfation, gamma-carboxylation of glutamic acid residues, hydroxylation, and ADP-ribosylation, all of which are described in basic texts, such as Proteins—Structure and Molecular Properties, 2nd Ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al. (Meth. Enzymol 182: 626-646(1990)) and Rattan et al. (Ann. N.Y. Acad. Sci. 663:48-62 (1992)). Other less common, but known modifications include acetylation, acylation, amidation, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, and sulfation.

In addition, other molecules made by the ICs can serve as readouts. Examples are lipids, sugars, and small molecules, e.g., steroid and peptide hormones.

In the case of the detection and/or measurement of non-protein molecules, or post translational modifications of proteins, the methodology involves incubation of the ICs with a body fluid, followed by a fractionation or verification step, followed by a detection step.

For example, if phosphorylation of a particular protein constitutes the readout, phosphorylation can be determined by any of several means. One method employs an antibody that is specific for the protein of interest, either in the phosphorylated or the unphosphorylated state. The antibody can be used to purify the phosphorylated or unphosphorylated protein of interest using a standard method, e.g., an immunoaffinity column.

The phosphorylation state of purified proteins can be determined using well known techniques that provide a sensitive measure of changes in the phosphorylation state of a protein of interest (e.g., mass-spectrometry, in which changes to the phosphorylation state can be detected due to changes in negative charge at physiological pH and changes in the charge to mass ratio of the protein).

Another technique for examining post-translational alterations in proteins consists of providing a labeled precursor, e.g., a radio-labeled phosphate moiety (e.g., ³²P-ATP) or lipid group (radio-labeled glycans (e.g., mannose)), which can then be incorporated into the protein of interest in ICs, providing a means of detecting the protein of interest due to incorporation of a labeled phosphate moiety, or a labeled lipid group that is added to the protein during glycosylation. The proteins of the ICs can be fractionated and separated by 2-D gel electrophoresis. The radioactive band pattern that results when the ICs are incubated with the test fluid is compared with the radioactive band pattern resulting from ICs that were not so incubated. This technique detects the phosphorylation that occurs as a result of the interaction of the IC with a molecule present in the body fluid.

Applications

The methods of the invention have a number of important applications, including the following.

Disease Diagnosis, Prognosis, and Treatment Monitoring

A patient who has been diagnosed with a disease, e.g., metastatic cancer, has a body fluid sample taken, and one or more appropriate ICs are incubated with the sample. For example, where the patient has metastatic cancer or another disease involving angiogenesis, ICs can be endothelial cells, used to gauge the angiogenic profile of a patient's blood. A 10 cm dish containing human umbilical vein endothelial cells (HUVEC) at, e.g., passage 3, at a density of approximately 10⁶ or 10⁷ cells/dish, are maintained in EGM2-MV medium (Clontech, Palo Alto, Calif.) that contains endothelial basal medium (EBM)-2 supplemented with 5% fetal bovine growth serum, gentamicin, amphoteracin B, hydrocortisone, ascorbic acid, and the growth factors VEGF, bFGF, hEGF, and IGF-1. The body fluid (e.g., blood) is added to the cells such that it constitutes approximately 50% of this medium (1:1 v/v ratio), and the cells are incubated with the fluid for approximately 4 hours, a time frame over which gene expression can be expected to change, if the fluid contains a substance that effects such a change. Following incubation, the RNA is extracted from the IC by standard methods and applied to suitable nucleic acid chips (e.g., Affymetrix chips), and an expression profile is obtained. A description of the use of nucleic acid chips can be found in, e.g., U.S. Pat. Nos. 6,344,316, 6,340,565, 6,333,155, 6,306,643, 6,040,138, 5,695,937, 5,445,934, and PCT Application Nos. WO 97/10365 and WO 92/10588.

The chip can be an off-the-shelf chip that contains a genome or specific portion of a genome, as described above. Alternatively, the extracted RNA from the IC can be applied to a chip containing a disease specific array (DSA), constructed as described above.

The patient is then treated and, following treatment, another sample of the same fluid is obtained, controlling for time of day and other factors, and again incubated with the IC to generate a second expression profile. Changes in the profile are indicative of the efficacy of treatment, or worsening of the condition, while a lack of change can be indicative that the therapy has thus far been ineffective.

In the methods of the invention, several different ICs from an IC panel can be used, and nucleic acid chips can be employed that contain a large number, including even the entire known genomic complement of genes from the human, mouse, or other genome. The chip species preferably are matched with the species from which the ICs were derived.

The process of monitoring and estimating the prognosis of a disease can be by providing a base-line expression pattern that has been generated by the incubation of the body fluid from non-diseased patients with the same ICs. A significant number of “normal” body fluid samples can be analyzed in this way (on the order of 50-100), permitting a relatively small subset, or cluster, of genes to emerge that have significant diagnostic, prognostic, and/or therapeutic monitoring value. Thus, a cluster of genes can be identified in this manner that are characteristically over-expressed in certain ICs when contacted with a body fluid obtained from patients suffering froma particular disease, e.g., urine from men with prostate cancer. Those genes are isolated and placed on substrates to form arrays, which, as is mentioned above, are referred to as disease-specific arrays (DSAs). Once a group of genes have been identified that are specific for a particular disease, the DSAs can be generated using these genes in full-length form (cDNA or genomic DNA), or fragments can be used, e.g., oligomers of 40-100 base pairs in length can be used, or multiple shorter oligomers within the sequence of each gene can be used (see, e.g., U.S. Pat. No. 6,261,776). The genes and/or oligonucleotides are applied to a substrate, e.g., a glass chip, and the signals generated by incubation with the body fluid are observed as indicators of the molecular signature of the disease or the molecular signature of genes that respond in patients in response to treatment.

Many different types of genes can potentially be included in a given DSA. Two types of genes are particularly preferred. The first type includes genes that are important in the diagnostic aspect of the disease. The second type includes genes that are known to change in expression when the disease successfully responds to one or more therapies or when the disease worsens. Another preferred class of genes on a DSA are those that change relatively early in the course of treatment, and that therefore can have their expression levels monitored to monitor the response of a patient to therapy.

Appropriate parameters for each DSA can be determined routinely by one skilled in the art using information provided by the companies that sell oligonucleotides for chip applications, and by using information generally known in the field of nucleic acid hybridization. These parameters include, for example, hybridization temperature, buffer components, and length of hybridization time.

Other routine parameters can be adjusted to optimize the changes in gene expression that are observed. These include increasing the time that the ICs incubate with the body fluid to allow various different genes to be regulated or to allow gene expression patterns to be identified. The conditions of the medium in which the cells are incubated can also be adjusted (e.g., by changing the basal cell media in which the cells are grown). For example, when using endothelial cell ICs, it can useful to omit or reduce the amount of VEGF or FGF in the medium so that the ICs will be more sensitive to smaller amounts of these molecules present in the patient's fluid. Another factor that can be altered is the percentage of the patient's fluid that is incubated with the ICs. One can also also include cycloheximide in the medium, which inhibits translation, thereby blocking the production of secondary gene products as well as reducing negative feedback of gene products that negatively regulate their own expression. Thus, the presence of cycloheximide in the medium might significantly enhance the level of gene expression over the incubation period. This strategy has been used successfully in previous work (see, e.g., Sukhatme et al., Oncogene Research 1:343-355, 1987).

Optimization of Treatment

The methods of the invention can be used to optimize functional drug levels and activities. A patient diagnosed with a particular disease has a body fluid sample taken, and a baseline determination is made. The patient is then subjected to an appropriate therapeutic regimen. During the course of treatment, additional body fluid samples are taken and compared to the baseline sample. These additional body fluid samples may be taken hourly, daily, weekly, or monthly. Using this information and the knowledge generally available in the art related to the administration of the therapeutic agent, one can increase or decrease the dose of the therapeutic agent to provide a higher functional drug level or activity, which is determined to be effective in the treatment of the particular disease.

The dosage may vary depending upon the dosage form employed and the route of administration utilized. The exact formulation, route of administration and dosage can be chosen by the individual physician in view of the patient's condition. See, e.g., Fingl et al., 1975, in “The Pharmacological Basis of Therapeutics”, Ch. 1 p. 1. Dosage amount and interval may be adjusted individually to provide levels of the agent that are sufficient to maintain the appropriate concentration. The amount of composition administered will, of course, be dependent on the subject being treated, on the subject's weight, the severity of the affliction, the manner of administration, and the judgment of the prescribing physician.

An example of this aspect of the invention is the monitoring of the effectiveness of anti-angiogenesis therapy administered to a patient diagnosed with cancer.

### EXAMPLE

### Monitoring Treatment of Ovarian Cancer

To monitor the treatment of a patient diagnosed with ovarian cancer, the physician, prior to initiating treatment, collects a sample of urine or of the fluid that typically bathes the lesions of the ovarian cancer that have metastasized to the peritoneum. The urine or peritoneal fluid is then processed as follows. Debris and cells are first removed by centrifugation at low speed. The supernatant is then passaged through a Whatman coarse filter to remove any additional microscopic debris, and then incubated with two or more different types (e.g., an endothelial cell and an epithelial cell). An endothelial cell IC, such as the HUVEC described above, provides information regarding the angiogenic profile of the fluid, and an epithelial cell IC, e.g., from an ovarian cancer cell line or of non-cancerous ovarian origin, responds to molecules generated by the patient's ovarian cancer cells, e.g., molecules that act in an autocrine manner. For example, the patient's ovarian cancer cells can be producing high levels of epidermal growth factor (EGF), and the chosen ovarian cancer cell line IC will respond to EGF by altering its gene expression profile. Following incubation of the ICs with the peritoneal fluid, changes in the gene expression profile of the ICs is determined, as described above. Changes in the gene expression profile, determined by comparing the gene expression profile obtained at the time of diagnosis to the gene expression profile obtained following therapy, is an indication of the effectiveness of the chosen therapy.

As is mentioned above, it is not always necessary to determine the expression pattern of multiple genes. Where the IC is an engineered cell containing a molecule encoded by a recombinant gene, such as a receptor or a transcription factor likely to be sensitive to a substance contained in a body fluid, the body fluid can be incubated with that engineered cell, and the interaction between disease-specific substances in the body fluid and the engineered molecule can be determined or measured, e.g., by a reporter molecule associated with the responsive molecule in the engineered IC.

All publications and patent applications mentioned in this specification are herein incorporated by reference to the same extent as if each independent publication or patent application was specifically and individually indicated to be incorporated by reference.

What is claimed is:

1. A method of obtaining information regarding a health parameter of a patient, said method comprising the steps of:
a) obtaining a sample of a body fluid from said patient;
b) ensuring that said sample is substantially cell-free;
c) incubating said sample with a cell; and
d) determining or measuring a property of said cell following incubation.

2. The method of claim 1, wherein step d) comprises determining the level of expression of one or more genes in said cell.

3. The method of claim 1, further comprising the steps of:
e) repeating steps a)-d) at a second time point; and
f) comparing the results of step d) for the two time points.

4. The method of claim 3, wherein the first sample is obtained prior to treatment of the patient for a medical disorder, and the second sample is obtained from the patient following treatment.

5. The method of claim 2, wherein step d) involves determining the level of expression of multiple genes in said cell.

6. A method of obtaining information regarding a health parameter of a patient, said method comprising the steps of:
a) obtaining a sample of a body fluid from said patient;
b) ensuring that said sample is substantially cell-free;
c) incubating said sample with a genetically engineered cell comprising a molecule that is responsive to a substance whose presence or absence in a body fluid is associated with a disease state; and
d) determining the response of said responsive molecule of said engineered cell to incubation with said body fluid.

7. The method of claim 6, wherein said responsive molecule is a receptor.

8. The method of claim 6, wherein said responsive molecule is a transcription factor.

9. The method of claim 6, wherein said responsive molecule is associated with a reporter molecule.

10. A method of making a disease-specific array (DSA) for use in the diagnosis or monitoring of a pre-determined disease, said method comprising the steps of:
a) obtaining a sample of a substantially cell-free body fluid from a patient suffering from said disease,
b) incubating said sample with a cell,
c) determining the level of expression of multiple genes in said cell following incubation.
d) comparing the information obtained in step c) with the expression level of multiple genes observed when the same cell type is incubated, under the same conditions, using a matched sample of the same body fluid obtained from a person suffering from said disease, to identify one or more genes that are expressed at different levels in the two samples, and
e) incorporating the genes identified in step d), or RNA molecules corresponding to said genes, or fragments of said genes, into or onto a carrier to form said DSA.

11. A DSA formed by the process of claim 10.

12. A method of obtaining a gene expression signature specific for a predetermined disease, said method comprising the steps of:
a) obtaining a sample of substantially cell-free body fluid from a patient suffering from said disease,
b) incubating said sample with a cell, and
c) determining the level of expression of multiple genes following incubation, such determination constituting said disease-specific signature.
